(19) United States
(12) Patent Application Publication
(10) Pub. No.: US 2021/0020279 A1
(43) Pub. Date: Jan. 21, 2021

(54) Computer-Automated Cannabidiol Product Recommendation System and Method

(71) Applicant: Smart Decision Inc., Boca Raton, FL (US)

(72) Inventors: Adam Green, Boca Raton, FL (US); Eric Gutmann, Boca Raton, FL (US); Benjamin Bau, Cambridge, MA (US); Savic Rasovic, Cambridge, MA (US)

(21) Appl. No.: 16/932,754

(22) Filed: Jul. 18, 2020

Related U.S. Application Data
(60) Provisional application No. 62/876,106, filed on Jul. 19, 2019.

(51) Int. Cl. G16H 10/60; G16H 20/10; G16H 70/40; A61K 31/047

(52) U.S. Cl. CPC G16H 10/60; A61K 31/047; G16H 70/40; G16H 20/10

(57) ABSTRACT

A computer-based system and method uses input received from a user about an ailment of the user and other characteristics of the user, and automatically recommends a CBD product, including a type and dosage, to the user. The system uses information about products and dosages used by other similar users and guidelines from medical professionals to produce the product recommendation for the user. The system may use machine learning to generate its recommendations and to improve its recommendations over time, based on feedback from users, medical professionals, and others about the efficacy of particular CBD products for treating particular ailments in various user cohorts.

### BACKGROUND

Cannabidiol (CBD) has become increasingly popular for addressing a wide variety of ailments, including chronic pain, anxiety and depression, epilepsy, cancer, acne and other skin issues, high blood pressure, addiction, and diabetes. Governments, however, have not yet published guidelines regarding the type and/or dose of CBD that should be used for various ailments. As a result, consumers typically must engage in trial and error when choosing the type and dose of CBD to use based on their particular needs. Furthermore, both healthcare professionals and manufacturers of CBD products tend to leave decisions about type and dosing to the consumer. As a result, although consumers have a significant need that could potentially be satisfied by CBD, they are left without a solution to the problem of selecting a type and dose of CBD to use for their own ailments.

### SUMMARY

A computer-based system and method uses input received from a user about an ailment of the user and other characteristics of the user, and automatically recommends a CBD product, including a type and dosage, to the user. The system uses information about products and dosages used by other similar users and guidelines from medical professionals to produce the product recommendation for the user. The system may use machine learning to generate its recommendations and to improve its recommendations over time, based on feedback from users, medical professionals, and others about the efficacy of particular CBD products for treating particular ailments in various user cohorts.

Other features and advantages of various aspects and embodiments of the present invention will become apparent from the following description and from the claims.

### BRIEF DESCRIPTION OF THE DRAWINGS

FIG. 1 is a dataflow diagram of a system for making recommendations to a user in relation to CBD use according to one embodiment of the present invention.

FIG. 2 is a flowchart of a method performed by the system according to one embodiment of the present invention.

### DETAILED DESCRIPTION

Several years ago, Israeli scientists documented the superior therapeutic properties of CBD-rich cannabis extract, compared to single-molecule cannabidiol (CBD). While the study compared “botanical preparations” to “pure single-molecule compounds,” a significant finding of the article was the following: “Healing was only observed when CBD was given within a very limited dose range, whereas no beneficial effect was achieved at either lower or higher doses.”¹ As this study indicates, it is critically important for a dose of CBD in an appropriate range to be administered to a person with an ailment if the CBD is to have a beneficial effect of that ailment. Embodiments of the present invention may be useful in identifying, recommending, and dispensing CBD in appropriate dose ranges in order to address this need, including using computer-automated systems and methods to performing some or all such functions. ¹ https://www.projectcbd.org/science/single-compound-vs-whole-plant-cbd.

Before describing the operation of various embodiments of the present invention in detail, various medical conditions that may be ameliorated by the appropriate use of CBD include, but are not limited to:
Pain: CBD may help to lower inflammation levels and pain perception. A study published in 2017 in Cannabis and Cannabinoid Research suggests that CBD might be useful as a pain therapy in place of opioids.
Anxiety & Depression: CBD may help with PTSD, generalized anxiety disorders, obsessive-compulsive disorder, and seasonal affective disorder.
Epilepsy: CBD may help to reduce the number of seizures. (In fact, this is the one condition that the FDA has approved an oral CBD formulation for LGS (Lennox-Gastaut Syndrome) & Dravet Syndrome.
Symptoms related to cancer treatment: The focus on CBD for cancer treatment has been for its use in reducing nausea and vomiting that often accompanies chemotherapy and radiation treatments. Researchers at the American Cancer Society have also discovered that CBD may slow the growth of cancer cells.
Acne and other skin issues: Researchers believe that topical CBD may be a potent antiacne agent, likely due to its anti-inflammatory properties. Studies have also found that CBD can be useful for reducing the itch and inflammation associated with eczema and psoriasis.
High Blood Pressure: Researchers in England have found that even a single dose of CBD may reduce resting blood pressure, which may ultimately reduce the risk of stroke. They concluded that the response may be due to CBD's anxiety-reducing (anxiolytic) and pain-reducing (analgesic) effects.
Addiction: CBD has shown promise in fighting addiction to everything from opioids and cocaine to alcohol and tobacco.
Diabetes: The American Journal of Pathology has suggested that CBD may lower fasting insulin levels and measures of insulin resistance.

Consumers also currently are known to purchase CBD in connection with the following ailments:
Sleep related disorders: CBD may promote relaxation and quality sleep when used as a sleep aid.
Burning fat and reducing obesity: CBD may help to lower body mass index by helping the body to burn fat.
Promote healthier heart: CBD may help to prevent arterial blockage, oxidative stress, and high blood pressure.
Arthritis: CBD has anti-inflammatory and pain relieving properties.
Anti-Spasmodic: CBD may help to suppress muscle spasms.
Bone stimulant: CBD may help to promote bone growth.
Anti-Psychotic: CBD may be tranquilizing in treating psychosis.
Neuroprotective: CBD may protect against nervous system degeneration.
Intestinal Anti-Prokinetic: CBD may reduce contractions in the small intestine.
Nausea & Vomiting: CBD may reduce symptoms of nausea and vomiting (not only in cancer patients).

Consumers also currently are purchasing CBD for use with ailments of their pets (e.g., cats and dogs), and embodiments of the present invention may be used to generate recommendations, e.g., for type and dosage of CBD, for pets. Examples of ailments that consumers currently are purchasing CBD in connection with for their pets include reducing anxiety, fear, and depression; reducing arthritis and joint pain; addressing symptoms of cancer; helping with glaucoma; reducing aggressive behavior; treating inflammatory bowel disease; improving coat and skin conditions; helping with nausea; and helping with seizures or epilepsy.

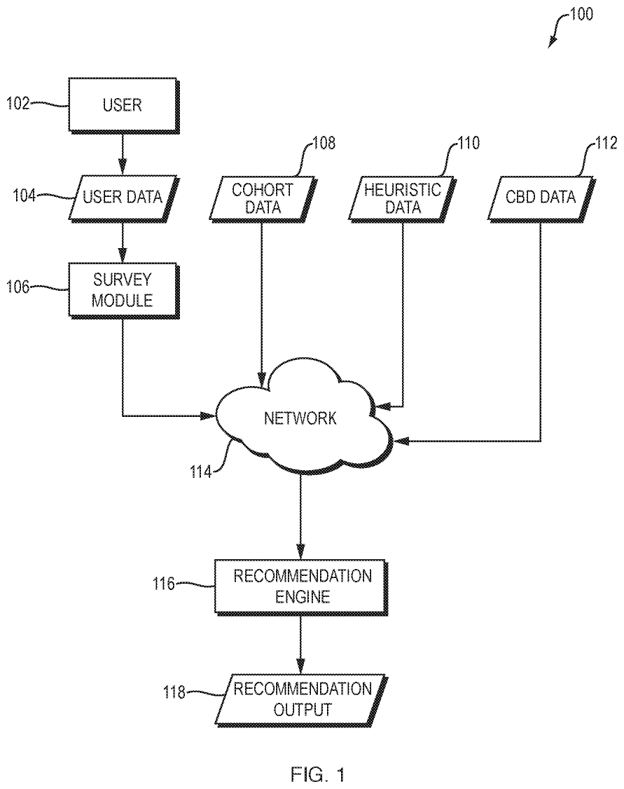

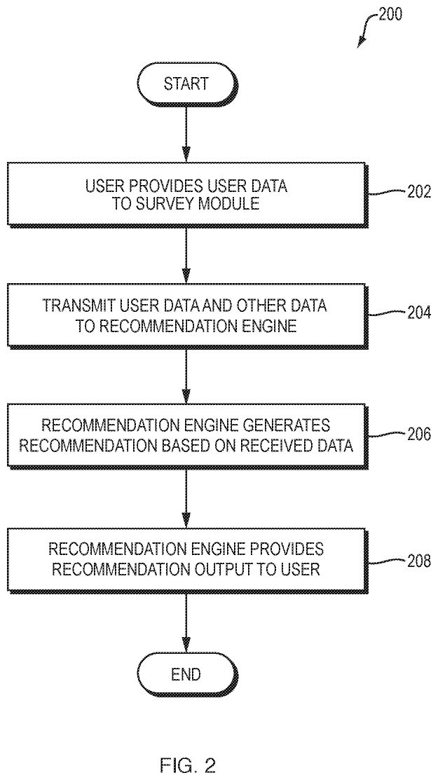

Referring to FIG. 1, a dataflow diagram is shown of a system 100 for making recommendations to a user in relation to CBD use according to one embodiment of the present invention. Referring to FIG. 2, a flowchart is shown of a method 200 performed by the system 100 according to one embodiment of the present invention.

The system 100 includes a user 102, who may be any person. Although in certain examples herein the user 102 is a person who has an ailment or is otherwise searching for and interested in purchasing one or more CBD products, these are merely examples and not limitations of the present invention. For example, the user 102 need not be a person who has an ailment or who is otherwise seeking to purchase a CBD product. Alternatively, for example, the user 102 may be an actual or potential distributor or seller of CBD products, a person who is seeking to purchase or obtain information about CBD products on behalf of another person, or a doctor or medical professional who is seeking to prescribe or recommend CBD products to a patient or other person. More generally, the user 102 may be any person.

The system also includes a survey module 106, which may be implemented on one or more computers in any of the ways disclosed herein. The user 102 provides input, referred to herein as user data 104, to the survey module 106 (FIG. 2, operation 202). The user 102 may provide the user data 104 as input using any one or more input devices, such as any of the input devices disclosed herein. The resulting user data 104 may be received, generated, and/or stored within a computer in any of the ways disclosed herein. The user data 104 may, for example, include one or more units of data that are descriptive of the user 102, such as data representing any one or more of the following, in any combination:
one or more ailments from which the user 102 is suffering, or believed or suspected to be suffering, or has suffered in the past;
a pain level experienced by the user 102 (e.g., on a scale of 1-10, or using any other quantity representing a measure of the pain level experienced by the user 102, such that the user's pain level may be measured as greater than or less than other pain levels);
one or more medications which the user 102 currently is taking, has purchased, and/or has been prescribed (where such medications may be represented by one or more of a medication manufacturer, brand name, generic name, type, or other identifier), and corresponding dosage(s) of such medication(s) (where each such dosage may be represented by any quantity that may be measured as greater than or less than other dosages);
demographic information about the user 102, such as any one or more of the user's age, sex, and postal code; and
personally identifying information about the user 102, such as any one or more of the user 102's name, mailing address, social security number and/or other unique identifier, email address, and username.

The module 106 is referred to herein as a “survey” module 106 because the module 106 may, for example, obtain the user data 104 by conducting a survey with the user 102, such as by providing (e.g., over the Internet or via a kiosk) the user 102 with one or more questions, in response to which the user 102 may provide input representing answers to such questions. The user data 104 may include such answers from the user 102. The survey module 106 need not, however, provide the user 102 with a survey or receive the user input 104 via a survey. More generally, the module 106 may be considered to be a user input module, which may obtain the user data 104 in any manner, such as by receiving text, audio (e.g., voice), image, and/or video input from the user 102, in any combination, whether or not such input is provided by the user 102 in response to one or more questions. The user 102 may provide input representing the user data 104 in a single unit of data (e.g., a single email message or web form), or in multiple units of data (e.g., multiple email messages or a series of web forms) over any amount of time. Embodiments of the present invention may update the user data 104 to reflect additional input received from the user 102 over time and thereby to produce updated user data 104.

Furthermore, the user data 104 may include (in whole or in part) data descriptive of the user 102 and/or of a user (not shown) other than the user 102. For example, the user 102 who provides the user data 104 may be a doctor or other healthcare professional who provides the user data 104, where the user data 104 is descriptive of a patient, rather than of the doctor.

The system 100 may include a variety of other data. For example, the system 100 may include cohort data 108, which may include data descriptive of one or more users, which may or may not include the user 102 (referred to herein as a “cohort”). The term “cohort” refers herein to a group of people who have some set of characteristics that have been determined to be sufficiently similar to each other according to some set of criteria. Such characteristics may, for example, include any one or more of ailments, pain levels, test results, clinical outcomes, test outcomes, demographic data (e.g., any one or more of sex, age, and nationality), and clinical data. For example, the cohort data 108 may include data descriptive of any of the properties of the user 102 described above, for each of one or more users (which may or may not include the user 102). For example, the cohort data 108 may include data descriptive of ailments and current medications taken by a plurality of users (which may or may not include the user 102). The cohort represented by the cohort data 108 may, for example, be a plurality of users who are similar in some way to the user 102, such as may be measured by demographic information such as age, sex, ethnicity, and/or geographic location.

The cohort data 108 may, for example, include data about the user 102 that is not included in the user data 104. For example, the cohort data 108 may include information about previous medical procedures (e.g., surgeries) performed on the user 102, even if the user data 104 does not include any information about such medical procedures. The system 100 may obtain such information for inclusion in the cohort data 108 from, for example, an Electronic Health Record (EHR) associated with the user 102.

The system 100 may also include heuristic data 110. As will be described in more detail below, the heuristic data 110 may, for example, include data representing any one or more of the following, in any combination:
one or more algorithms which may be applied to any data described herein to produce recommendation output 118, as that term is used herein;
one or more rules which may be applied to any data described herein to produce recommendation output 118; and
one or more machine learning methods, such as neural networks and/or genetic algorithms, which may be applied to any data described herein to produce recommendation output 118.

More generally, and as the description herein makes clear, the heuristic data 100 may be implemented in any of a variety of ways to enable the recommendation engine 116 to generate recommendation output 118 based on any of the data disclosed herein.

The system 100 may also include CBD data 112. The CBD data 112 may include, for example, data representing any one or more of the following, in any combination:
Names, product identifiers (e.g., SKUs), and/or quantities of specific CBD products.
One or more recommended dosages of each of one or more such CBD products, where such recommended dosages may include more than one recommended dosage for any particular CBD product. For example, one recommended dosage for a particular CBD product may apply to one cohort (e.g., combination of age and sex), while another recommended dosage for the same CBD product may apply to another cohort (e.g., a different combination of age and sex).
One or more actual dosages applied of such CBD products, where such actual dosages may include more than one actual dosage for any particular CBD product. For example, one actual dosage for a particular CBD product may represent an average actual dosage of that particular CBD product used by one cohort (e.g., combination of age and sex), while another actual dosage for the same CBD product may represent an average actual dosage of that particular CBD product used by another cohort (e.g., a different combination of age and sex).
Outcomes achieved by such CBD products, such as how well certain dosages worked when applied by people within a particular cohort.

The system 100 may provide (e.g., transmit) some or all of the user data 104 (which may be output, possibly in a processed form, by the survey module 106), cohort data 108, heuristic data 110, and CBD data 112 to a recommendation engine 116, such as by transmitting such data to the recommendation engine 116 over a network 114, such as the Internet (FIG. 2, operation 204). The recommendation engine 116 receives the provided data, and generates recommendation output 118 based on some or all of the received data (FIG. 2, operation 206). The recommendation engine 116 may, for example, apply the operations (e.g., algorithms) specified by the heuristic data 110 to some or all of the user data 104, cohort data 108, and CBD data 112 to generate the recommendation output 118.

The recommendation engine 116 may provide the recommendation output 118 to the user 102 (FIG. 2, operation 208). For example, the recommendation engine 116 may transmit the recommendation output 118 over the network 114 to the user 102, such as by transmitting the recommendation output 118 over the network 114 to a computer associated with the user 102. The system 100 (e.g., the computer associated with the user 102) may output (e.g., display) some or all of the recommendation output 118 to the user 102, such as by displaying, printing, or otherwise generating output representing a type and/or dosage of CBD represented by the recommendation output 118.

The recommendation output 118 may include any of a variety of data representing a recommendation for a CBD product, such as any one or more of the following information about the product, in any combination:
a name of the product;
a unique product identifier (such as a stock keeping unit (SKU));
a recommended dosage of the product;
a quantity of the product (which may be greater than the recommended dosage of the product), such as the quantity contained within a particular unit (e.g., bottle, box, or other packaging) of the product; and
an indication of whether the product is edible or topical. Non-limiting examples of forms that the product may take include drinks, capsules, and oils.

The recommendation output 118 may, for example, include some or all of one or more of the user data 104, cohort data 108, and CBD data 112. For example, the recommendation output may include, for a recommended CBD product, an average of the dosage used of that product by one or more users represented by the cohort data 108. In this way, embodiments of the present invention may inform the user 102 of the average dosage used by similar people (e.g., having similar ailments, pain levels, and/or ages) who use the CBD product that is recommended by the recommendation output 118. For example, if the user data 104 indicates that the user 102 has a particular ailment and the recommendation output 118 recommends that the user 102 use a particular CBD product, then the system 100 may identify a subset of the cohort data 108 representing other users who have the same ailment and who use the recommended CBD product, and inform the user 102 of the average dosage of the recommended CBD product used by those other users. This is merely one example of a way in which embodiments of the present invention may provide the user 102 with information about other similar users to assist the user 102 in using the recommended CBD product.

Once the recommendation engine 116 has generated the recommendation output 118, the system 100 may automatically dispense a unit of the product represented by the recommendation output. For example, if the user 102 provides the user data 104 to a kiosk, and that kiosk provides the recommendation output 118 to the user 102, then the kiosk may dispense a product having features that match those represented by the recommendation output 118. For example, the recommendation output 118 may include a unique product identifier, such as an SKU, in which case the kiosk may automatically select and dispense a unit of a CBD product having the SKU.

The recommendation engine 116 may, for example, use any of a variety of known recommendation algorithms to generate recommendation output 118, which represents one or more CBD products that are the same as or similar to CBD products previously purchased by users represented by the cohort data 108 (which may or may not include data representing the user 102). For example, if the user data 104 indicates that the user 102 has purchased one or more particular CBD products, then the recommendation engine 116 may, based on such data indicating that the user 102 has purchased such particular CBD products, generate recommendation output 118 which includes data representing one or more other particular CBD products (i.e., other than the particular products purchased by the user 102) that were purchased by users other than the user 102 in the cohort represented by the cohort data 108. The recommendation engine 116 may also take into account data indicating that the user 102 has liked (e.g., rated positively or highly) one or more products, and/or data indicating that users other than the user 102 within the cohort represented by the cohort data 108 have liked (e.g., rated positively or highly) one or more products. For example, if the user data 104 indicates that the user 102 has purchased and liked one or more particular CBD products, then the recommendation engine 116 may, based on such data indicating that the user 102 has purchased and liked such particular CBD products, generate recommendation output 118 which includes data representing one or more other particular CBD products (i.e., other than the particular products purchased by the user 102) that were purchased and liked by users other than the user 102 in the cohort represented by the cohort data 108.

As another example, instead of or in addition to using the techniques described above, the recommendation engine 118 may generate a profile (e.g., within the user data 104) associated with the user 102. The recommendation engine 116 may, for example, generate the profile based on other data in the user data 104, such as the user 102's answers to a survey, such as any of the surveys disclosed herein. The user 102's answers to such a survey may, but need not, include any information about CBD products purchased or used by the user 102. The recommendation engine 116 may, based on the user 102's profile and one or more profiles of other users (e.g., based on data in the cohort data about such other users), generate recommendation output 118 representing one or more CBD products to recommend to the user 102. For example, the profiles of the other users may include data representing one or more CBD products purchased, used, and/or liked by such other users. The recommendation engine 116 may, for example, identify the profile(s) of one or more users that are sufficiently similar to the profile of the user 102 (e.g., by determining that the user 102's profile and the profile(s) of the other user(s) satisfy some similarity criteria); identify one or more CBD products purchased, used, or liked by the user(s) associated with the identified profile(s); and then generate, within the recommendation output 118, data representing such identified CBD product(s) purchased, used, or like by the user(s) associated with the identified profile(s).

As yet another example, the recommendation engine 116 may receive, as input, data representing one or more reviews of a plurality of CBD products (referred to herein as “review data”). Such reviews may include, for example, text describing evaluations of such CBD products. The recommendation engine 116 may receive such data in any of a variety of ways, such as by automatically scraping data automatically from a plurality of web pages (such as e-commerce, news, and/or blog web pages) to generate scraped data. Non-limiting examples of web scraping toolkits that may be used to perform such scraping include Scrapy, PySpider, MechanicalSoup, and Puppeteer. The recommendation engine 116 may, however, receive such data from any source and using any technique(s).

The recommendation engine 116 may apply natural language processing (NLP) to a particular review within the review data and generate, as output, NLP output. The recommendation engine 116 may generate, based on the review data and/or the NLP output, data representing a profile of the author of the review (referred to herein as an “author profile”). Non-limiting examples of NLP toolkits that may be used to perform NLP include NLTK and Rosette.

Embodiments of the present invention may, for example, apply automated sentiment analysis to the review data and/or the NLP output, to generate sentiment data representing the sentiment(s) (e.g., positive and/or negative sentiments) expressed by the author of the review data. Non-limiting examples of sentiment analysis toolkits that may be used to perform such sentiment analysis include Rosette, Lexalytics, and Amazon Comprehend. The recommendation engine 116 may, for example, generate the data representing the author profile based on any combination of the review data, the NLP output, and the sentiment data.

As the above implies, the recommendation engine 116 may generate the author profile automatically and without receiving input from the review author or any other user. Such a profile may include a variety of data related to the review author, such as data representing predictions of one or more of: an ailment of the review author, a severity of that ailment, a name of the review author, an age of the review author, a geographic location of the review author, and a sex of the review author. Similarly, the recommendation engine 116 may apply NLP to the particular review and automatically generate a profile (referred to herein as a “product profile”) containing a variety of discrete data relating to the CBD product that is reviewed in the particular review, such one or more of the following: the manufacturer, brand name, generic name, type, and recommended dosage of the CBD product reviewed in the particular review. The recommendation engine 116 may repeat these steps for a plurality of product reviews to automatically generate review author profiles and product profiles based on and associated with the plurality of product reviews.

Once the recommendation engine 116 has generated one or more such author profiles, the system 100 may use such author profiles in any of the ways that the recommendation engine 116 uses the user profiles, as disclosed herein. For example, such an automatically-generated author profile may be provided as input to the recommendation engine 116, which may generate recommendation output 118, based on that author profile, containing data representing one or more recommended CBD products.

This embodiment is useful, for example, in situations in which there is no user-generated profile for a user, because the system 100 may instead automatically generate a profile of a user based on one or more product reviews written by the user, and then automatically generate product recommendations (within the recommendation output 118) for that user, thereby eliminating the need for the user to manually generate a user profile in order to receive product recommendations.

This embodiment is also useful, for example, to automatically generate a profile of a user based on one or more product reviews written by the user, and then to provide that profile as input to train the recommendation engine 116 in any of the ways disclosed herein. Such an automatically-generated profile for the user 102 may, for example, be added to (or used as) the user data 104 for the user 102. The system 100 may similarly automatically generated profiles for a plurality of users and add those profiles to the cohort data 108. The recommendation engine 116 may then use such automatically-generated profiles (e.g., within the user data 104 and/or cohort data 108) to generate the recommendation output 118 in any of the ways disclosed herein. For example, as described above, the recommendation engine 116 may implement a neural network, and the system 100 may train that neural network using the automatically-generated user profile(s) within the user data 104 and/or cohort data 108. The recommendation engine 116 may then use the resulting trained neural network to make recommendations for one or more users, using any of the techniques disclosed herein.

Although the above description states that the recommendation engine 116 may automatically generate a profile of a user based on a particular review written by that user, the recommendation engine 116 may similarly automatically generate the user's profile based on a plurality of reviews written by that user if, for example, it is known that the plurality of reviews were written by the same user. The same techniques disclosed herein may be used to automatically generate a user profile based on a plurality of reviews written by the user. Furthermore, the term “review,” as used herein, may refer to any data (e.g., text, audio, and/or video), such as any input received from a user, which describes or implies one or more of the user's opinions about one or more products. For example, embodiments of the present invention may treat an email message written by a user as a “review” if that email message contains text representing opinions of the user about a product, even if that email message was not written with the intent of publishing it as a review of the product. Note that, as in the example of web scraping, the system 10 may receive a review authored by a user even if the user does not provide that review as input to the recommendation engine 116 or to the system 100 more generally. For example, the user may provide the review to an external computer system (e.g., an email server or a web server), i.e., a computer system that is external to the system 100 and that does not include the recommendation engine 116, and the system 100 may, at a subsequent time, automatically obtain (e.g., pull) the review as input from that external computer system, such as by performing web scraping, even if the user did not provide input indicating that the review should be provided as input to the system 100.

In some embodiments of the present invention, the recommendation engine 116 may generate and include, in the recommendation output 118, data representing a first CBD product based on at least one review of a second, different, CBD product. In certain embodiments of the present invention, the recommendation engine 116 may generate and include, in the recommendation output 118, data representing a first CBD product based on at least one review of a second, different, CBD product, and not based on any reviews of the first CBD product.

For example, consider that CBD products typically consist of combinations of ingredients in certain concentrations (include both active ingredients and inactive ingredients). Embodiments of the present invention may treat a review of a first product as being applicable to a second product if the first and second products have similar combinations of ingredients. The recommendation engine 116 may then generate a recommendation, within the recommendation output 118, for the second product based on one or more reviews of the first product.

More specifically, the recommendation engine 116 may identify one or more reviews of a first product. The recommendation engine 116 may determine that the first product is sufficiently similar to a second product, e.g., that a combination of ingredients of the first product is sufficiently similar to a combination of ingredients of a second product. In other words, the recommendation engine 116 may determine that the first product and the second product satisfy a similarity criterion. For example, the recommendation engine 116 may determine that the first product is sufficiently similar to the second product in response to determining that the first and second products share at least some minimum number or percentage of ingredients in common. As yet another example, the recommendation engine 116 may determine that the first product is sufficiently similar to the second product in response to determining that the first and second products were manufactured by the same manufacturer and/or are associated with the same brand name.

In response to determining that the first product is sufficiently similar to the second product, the recommendation engine 116 may use any of the techniques disclosed herein to generate a recommendation, within the recommendation output, that the user 102 use the second product, based on data related to the first product, such as one or more reviews of the first product. In this way, the recommendation engine 116 may effectively treat the second product as if it had received the same reviews as the first product, even if it did not receive those reviews. In fact, the recommendation engine 116 may generate a recommendation, within the recommendation output 118, to use the second product, based on input (e.g., reviews of the first product) that does not include any reviews of the second product.

As another example, the recommendation engine may generate a recommendation to use the second product based on both reviews of the first product and reviews of the second product, in response to determining that the first and second products are sufficiently similar to each other. Such embodiments may be particularly useful if, for example, there are a large number of reviews of the first product but only one or a small number of reviews of the second product.

At any point after receiving the recommendation output 118, the user 102 may provide feedback data (not shown) to the system 100. Such feedback data may include any of a variety of data representing feedback by the user 102 relating to the CBD product recommended by the recommendation output 118. For example, the feedback data may include any one or more of the following in any combination:
the actual dosage of the CBD product used by the user 102, such as the actual dosage(s) used at one or more particular times and/or an average dosage used over a particular period of time;
a pain level experienced by the user 102 after using the recommended CBD product and/or a relative reduction in pain level experienced by the user 102 after using the recommended CBD product (e.g., a −3 representing a decrease of 3 in the pain level experienced by the user 102); and
any other measure of the effectiveness of the recommended product in addressing the user 102's ailment.

The system 100 may update the cohort data 108 to include the feedback data from the user 102 in any of a variety of ways, such as by adding the feedback data to the cohort data 108 and/or modifying the cohort data 108 to reflect the feedback data. The system 100 may apply any kind of machine learning, predictive analytics, and/or artificial intelligence to update the cohort data 108 and/or heuristic data 110 based on the feedback data received from the user 102. For example, the heuristic data 110 may be or include a neural network, and the system 100 may train or update the training of the neural network based on the cohort data 108. As a result, when the recommendation engine 116 next produces recommendation output, based on additional user data 104 from the same user 102, or based on user data received from another user, the recommendation engine 116 may produce the new recommendation output based on the updated heuristic data 110 (e.g., neural network) and thereby produce improved output. The system 100 may continue to improve itself over time based on additional information obtained from the user 102, other users, and other sources.

The heuristic data 110 may be pre-configured (e.g., manually) to include certain rules, heuristics, and/or algorithms. For example, the heuristic data 110 may be pre-configured not to produce recommendation output 118 representing recommendations which are contraindicated by the user 102's user data 104. For example, the heuristic data 110 may be pre-configured not to produce recommendation output 118 representing recommendations for a product, or for a dosage of a product, which would likely be harmful to the user 102. As a particular example, the heuristic data 110 may be pre-configured not to produce recommendation output 118 representing a recommendation that the user 102 use a CBD product in an inhaled form if the user data 104 indicates that the user 102 has a respiratory illness (e.g., lung cancer).

The following is one specific example of a survey that the survey module 106 may provide to the user 102 to elicit the user input 104. In the following example, each survey question is provided, followed by the possible answers to that question, followed by the answer provided by the user. The user's answer may then influence the system 100's selection of the next question to ask the user 102. This particular type of survey is merely one example and does not constitute a limitation of the present invention.
Question: “What ailment are you looking for relief from?” Available answers: Anxiety including separation, Nausea, High Blood Pressure, Epilepsy, Anxiety/Depression, Acne, Pain. User's answer: Pain
Question: “From 1 (just a little bit of pain) to 10 (severe, unbearable pain), what would you say your pain level is?”. Available answers: 1, 2, 3, 4, 5, 6, 7, 8, 9, 10. User's answer: 8.
Question: “Which method of CBD do you prefer?” Available answers: Drops under the tongue/sublingual, Gummy/candy, Topical, Vape/Inhale. User's answer: Drops under the tongue.

The user 102's answers in the example above are an example of the user data 104 in FIG. 1. In this example, the heuristic data 100 may be configured to implement the following logic:
If the user 102 selects a pain level of 5 or higher, then recommend a dosage of 1000 mg of CBD.
If the user 102 selects a pain level of 3 or 4, then recommend a dosage of 500 mg of CBD.
If the user 102 selects a pain level of 1 or 2, then recommend a dosage of 200 mg of CBD.

The above example, in which the CBD dosage recommended by the recommendation output 118 is based solely on the pain level indicated by the user 102, is merely an example and does not constitute a limitation of the present invention.

Embodiments of the present invention may be implemented in any of a variety of ways. For example, embodiments of the present invention may be implemented within a website to enable the website to make recommendations to users of the website in accordance with the system 100 and method 200 disclosed herein. The website may, for example, receive user data 104 from a user of the website and, in response to receiving that user data 104, provide recommendation output 118 to the user in the manner disclosed herein. Such a website may take into account the actual inventory available to the website and only recommend products which are within that inventory.

Embodiments of the present invention may, for example, make use of automatic location detection technology. For example, embodiments of the present invention which are implemented on mobile communication devices (e.g., smartphones and tablets) may determine automatically that such a device is near or within a particular store and, in response to such a determination, only recommend products to the user which are within the current inventory of that particular store. As another example, embodiments of the present invention may use location identification technology (such as Global Positioning System (GPS) technology) to automatically identify a current location of the user 102.

Embodiments of the present invention have a variety of advantages. In particular, embodiments of the present invention may be used to automatically generate a recommendation to a person for a CBD product, such as a name and dosage of a product, based on information about the person, such as the person's ailment(s), pain level, age, and sex. This eliminates the guesswork that is so often involved in selecting and purchasing CBD products. Furthermore, the use of data obtained from other users of CBD products enables embodiments of the present invention to grow more intelligent and provide higher quality recommendations over time.

Embodiments of the present invention may be used to benefit a wide variety of people, such as:
Consumers: Embodiments of the present invention may be used to help consumers directly to select the right CBD product(s) (e.g., type and/or dosage) for their needs. A consumer who receives recommendation output from an embodiment of the present invention may use that output to select and purchase CBD in accordance with the recommendation output.
CBD Manufacturers: At present, most CBD manufacturers are selling their products directly to consumers. If such manufacturers could implement embodiments of the present invention, e.g., on their websites, they could provide consumers with the information and peace of mind necessary to assist such consumers in making an informed purchasing decision.
Medical Practices: Although medical practices (especially orthopedic practices) are starting to carry and sell CBD products from their offices, the doctors and other staff at such offices often do not have sufficient familiarity with CBD to recommend the right CBD products and/or dosages to their patients. Medical practices which implement embodiments of the present invention could use such embodiments to provide the right type and dosages of CBD to their patients.
Retailers and Other Online Aggregators: Businesses that sell (e.g., aggregate CBD products from multiple CBD manufacturers) would benefit from implementing embodiments of the present invention in order to enable consumers to identify the type and dosage of CBD product that they require based on their ailment and other information, so that such consumers could make a purchase without consulting a physician or other person.

It is to be understood that although the invention has been described above in terms of particular embodiments, the foregoing embodiments are provided as illustrative only, and do not limit or define the scope of the invention. Various other embodiments, including but not limited to the following, are also within the scope of the claims. For example, elements and components described herein may be further divided into additional components or joined together to form fewer components for performing the same functions.

Any of the functions disclosed herein may be implemented using means for performing those functions. Such means include, but are not limited to, any of the components disclosed herein, such as the computer-related components described below.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements will also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Various elements of the present invention may be implemented on various devices, and may communicate with each other over one or more computer networks. For example, the user 102 may input the user data 104 on a first computing device, such as a desktop computer, laptop computer, tablet computer, smartphone, or kiosk. The first computing device may transmit the user data 104 (or data derived therefrom) to a second computing device over the network 114. The second computing device (e.g., a server) may apply the heuristic data 110 to the received user data 104 and generate the recommendation output 118 based on the user data 104. The second computing device may transmit the recommendation output 118 to the first computing device over the network 114. This is merely one example of how embodiments of the present invention may be implemented using multiple computing devices in communication over a network.

Any data disclosed herein may be implemented, for example, in one or more data structures tangibly stored on a non-transitory computer-readable medium. Embodiments of the invention may store such data in such data structure(s) and read such data from such data structure(s).

What is claimed is:

1-16. (canceled)

17. A method performed by at least one computer processor executing computer program instructions stored on at least one non-transitory computer-readable medium, the method comprising:
(A) receiving data representing a plurality of opinions of a first product;
(B) determining that a combination of ingredients of a second product is similar to a combination of ingredients of the first product;
(C) in response to the determination of (B), generating, based on the data representing the plurality of opinions of the first product, recommendation data representing a recommendation that a first user use the second product.

18. The method of claim 17, wherein the data representing the plurality of opinions of the first product comprises data representing a plurality of reviews of the first product by a plurality of users.

19. The method of claim 17, wherein (B) comprises determining that the first product and the second product share at least a minimum number of ingredients in common.

20. The method of claim 17, wherein (B) comprises determining that the first product and the second product were manufactured by the same manufacturer.

21. The method of claim 17, wherein the data representing the plurality of opinions of the first product does not include any data representing an opinion of the second product.

22. The method of claim 17, wherein the data representing the plurality of opinions of the first product includes data representing an opinion of the first product and data representing an opinion of the second product.

23. A system comprising at least one non-transitory computer-readable medium having computer program instructions stored thereon, wherein the computer program instructions are executable by at least one computer processor to perform a method, the method comprising:
(A) receiving data representing a plurality of opinions of a first product;
(B) determining that a combination of ingredients of a second product is similar to a combination of ingredients of the first product;
(C) in response to the determination of (B), generating, based on the data representing the plurality of opinions of the first product, recommendation data representing a recommendation that a first user use the second product.

24. The system of claim 23, wherein the data representing the plurality of opinions of the first product comprises data representing a plurality of reviews of the first product by a plurality of users.

25. The system of claim 23, wherein (B) comprises determining that the first product and the second product share at least a minimum number of ingredients in common.

26. The system of claim 23, wherein (B) comprises determining that the first product and the second product were manufactured by the same manufacturer.

27. The system of claim 23, wherein the data representing the plurality of opinions of the first product does not include any data representing an opinion of the second product.

28. The system of claim 23, wherein the data representing the plurality of opinions of the first product includes data representing an opinion of the first product and data representing an opinion of the second product.
